SEQUENCE LISTING (83 sequences)

SEQ ID NO: 1 - DNA, 23 bp, synthetic construct
  source 1..23: mol_type = other DNA
agatcagtgcctactaatactgg

SEQ ID NO: 2 - DNA, 23 bp, synthetic construct
  source 1..23: mol_type = other DNA
ttaggtataccagtattagtagg

SEQ ID NO: 3 - DNA, 23 bp, synthetic construct
  source 1..23: mol_type = other DNA
ttgatgatgactaagcctaatgg

SEQ ID NO: 4 - DNA, 23 bp, synthetic construct
  source 1..23: mol_type = other DNA
agggaacttcaccccccatgagg

SEQ ID NO: 5 - DNA, 23 bp, synthetic construct
  source 1..23: mol_type = other DNA
aacttcaccccccatgaggaagg

SEQ ID NO: 6 - DNA, 23 bp, synthetic construct
  source 1..23: mol_type = other DNA
atatagctgctgctcatgagagg

SEQ ID NO: 7 - DNA, 23 bp, synthetic construct
  source 1..23: mol_type = other DNA
ctttggtagaaattgaccactgg

SEQ ID NO: 8 - DNA, 23 bp, synthetic construct
  source 1..23: mol_type = other DNA
gcttgaggcatatgttgttgtgg

SEQ ID NO: 9 - DNA, 23 bp, synthetic construct
  source 1..23: mol_type = other DNA
ctcatgagaggaacaatgtcagg

SEQ ID NO: 10 - DNA, 23 bp, synthetic construct
  source 1..23: mol_type = other DNA
acaaggataatatcctcctcagg

SEQ ID NO: 11 - DNA, 23 bp, synthetic construct
  source 1..23: mol_type = other DNA
acttcaccccccatgaggaaggg

SEQ ID NO: 12 - DNA, 23 bp, synthetic construct
  source 1..23: mol_type = other DNA
gaaaggtccatggacacctgagg

SEQ ID NO: 13 - DNA, 23 bp, synthetic construct
  source 1..23: mol_type = other DNA
tacatccaagaacatggtccagg

SEQ ID NO: 14 - DNA, 23 bp, synthetic construct
  source 1..23: mol_type = other DNA
attcatttgcaagctctactggg

SEQ ID NO: 15 - DNA, 23 bp, synthetic construct
  source 1..23: mol_type = other DNA
aatatctcaagactcttggaagg

SEQ ID NO: 16 - DNA, 23 bp, synthetic construct
  source 1..23: mol_type = other DNA
ctaaggccaggaatcaagagagg

SEQ ID NO: 17 - DNA, 23 bp, synthetic construct
  source 1..23: mol_type = other DNA
tccccaaagcaactcaacaaggg

SEQ ID NO: 18 - DNA, 23 bp, synthetic construct
  source 1..23: mol_type = other DNA
ttctggcatggaatcacaagtgg

SEQ ID NO: 19 - DNA, 23 bp, synthetic construct
  source 1..23: mol_type = other DNA
gtctcttacatccaagaacatgg

SEQ ID NO: 20 - DNA, 23 bp, synthetic construct
  source 1..23: mol_type = other DNA
tatcatcccttcctcatgggggg

SEQ ID NO: 21 - DNA, 23 bp, synthetic construct
  source 1..23: mol_type = other DNA
gcatcagactcaacagccagtgg

SEQ ID NO: 22 - DNA, 23 bp, synthetic construct
  source 1..23: mol_type = other DNA
tctcaagactcttggaaggttgg

SEQ ID NO: 23 - DNA, 23 bp, synthetic construct
  source 1..23: mol_type = other DNA
ttccccaaagcaactcaacaagg

SEQ ID NO: 24 - DNA, 23 bp, synthetic construct
  source 1..23: mol_type = other DNA
ggcatatgttgttgtggaagagg

SEQ ID NO: 25 - DNA, 23 bp, synthetic construct
  source 1..23: mol_type = other DNA
gatgaagatcatcagctccaagg

SEQ ID NO: 26 - DNA, 23 bp, synthetic construct
  source 1..23: mol_type = other DNA
aggtccatggacacctgaggagg

SEQ ID NO: 27 - DNA, 23 bp, synthetic construct
  source 1..23: mol_type = other DNA
ttgtggaagataggaagctatgg

SEQ ID NO: 28 - DNA, 23 bp, synthetic construct
  source 1..23: mol_type = other DNA
gtctgttctttgtggaagatagg

SEQ ID NO: 29 - DNA, 23 bp, synthetic construct
  source 1..23: mol_type = other DNA
ttatcatcccttcctcatggggg

SEQ ID NO: 30 - DNA, 23 bp, synthetic construct
  source 1..23: mol_type = other DNA
agaaccaaaacaatgctgcttgg

SEQ ID NO: 31 - DNA, 23 bp, synthetic construct
  source 1..23: mol_type = other DNA
agactgatgactaagcctaatgg

SEQ ID NO: 32 - DNA, 23 bp, synthetic construct
  source 1..23: mol_type = other DNA
tgggatgatgatcccttgagtgg

SEQ ID NO: 33 - DNA, 23 bp, synthetic construct
  source 1..23: mol_type = other DNA
atcttccccaaagccactcaagg

SEQ ID NO: 34 - DNA, 23 bp, synthetic construct
  source 1..23: mol_type = other DNA
tcttccccaaagccactcaaggg

SEQ ID NO: 35 - DNA, 23 bp, synthetic construct
  source 1..23: mol_type = other DNA
atgatatagctgctcatgagagg

SEQ ID NO: 36 - DNA, 23 bp, synthetic construct
  source 1..23: mol_type = other DNA
tggtggtggcgtagataacatgg

SEQ ID NO: 37 - DNA, 23 bp, synthetic construct
  source 1..23: mol_type = other DNA
agaaccttaacaatgctgcttgg

SEQ ID NO: 38 - DNA, 23 bp, synthetic construct
  source 1..23: mol_type = other DNA
tgaaagtgttggtcaccaagtgg

SEQ ID NO: 39 - DNA, 23 bp, synthetic construct
  source 1..23: mol_type = other DNA
aagtgttggtcaccaagtggagg

SEQ ID NO: 40 - DNA, 23 bp, synthetic construct
  source 1..23: mol_type = other DNA
tgatgctgagtgtggatccaagg

SEQ ID NO: 41 - DNA, 23 bp, synthetic construct
  source 1..23: mol_type = other DNA
gatgagcatcaagagggtggtgg

SEQ ID NO: 42 - DNA, 23 bp, synthetic construct
  source 1..23: mol_type = other DNA
aggcaaaaatctgagaacagtgg

SEQ ID NO: 43 - DNA, 23 bp, synthetic construct
  source 1..23: mol_type = other DNA
ggtgttataccagtattagtagg

SEQ ID NO: 44 - DNA, 23 bp, synthetic construct
  source 1..23: mol_type = other DNA
agtgcttgaggcatatgttgtgg

SEQ ID NO: 45 - DNA, 23 bp, synthetic construct
  source 1..23: mol_type = other DNA
tggctcttggatgaaagtgttgg

SEQ ID NO: 46 - DNA, 23 bp, synthetic construct
  source 1..23: mol_type = other DNA
tgaggcatatgttgtggaagagg

SEQ ID NO: 47 - DNA, 23 bp, synthetic construct
  source 1..23: mol_type = other DNA
aaagatgagcatcaagagggtgg

SEQ ID NO: 48 - DNA, 23 bp, synthetic construct
  source 1..23: mol_type = other DNA
aaacaaagatgagcatcaagagg

SEQ ID NO: 49 - DNA, 23 bp, synthetic construct
  source 1..23: mol_type = other DNA
aacaaagatgagcatcaagaggg

SEQ ID NO: 50 - DNA, 23 bp, synthetic construct
  source 1..23: mol_type = other DNA
gcatcagactcaacagcaagtgg

SEQ ID NO: 51 - DNA, 23 bp, synthetic construct
  source 1..23: mol_type = other DNA
agggaacttcactccccatgagg

SEQ ID NO: 52 - DNA, 23 bp, synthetic construct
  source 1..23: mol_type = other DNA
agcagcagcaacaataatcatgg

SEQ ID NO: 53 - DNA, 23 bp, synthetic construct
  source 1..23: mol_type = other DNA
actccatcatctcctccacttgg

SEQ ID NO: 54 - DNA, 23 bp, synthetic construct
  source 1..23: mol_type = other DNA
gagcatcaagagggtggtggtgg

SEQ ID NO: 55 - DNA, 23 bp, synthetic construct
  source 1..23: mol_type = other DNA
gatgaagatattcagctccaagg

SEQ ID NO: 56 - DNA, 23 bp, synthetic construct
  source 1..23: mol_type = other DNA
ctctcttgattcctggcctgagg

SEQ ID NO: 57 - DNA, 23 bp, synthetic construct
  source 1..23: mol_type = other DNA
aacttcactccccatgaggaagg

SEQ ID NO: 58 - DNA, 23 bp, synthetic construct
  source 1..23: mol_type = other DNA
attcacttgcaagctctactggg

SEQ ID NO: 59 - DNA, 23 bp, synthetic construct
  source 1..23: mol_type = other DNA
acaaggatgatatcctcctcagg

SEQ ID NO: 60 - DNA, 23 bp, synthetic construct
  source 1..23: mol_type = other DNA
attatcattccttcctcatgggg

SEQ ID NO: 61 - DNA, 28 bp, synthetic construct
  misc_feature 1..28: note = primer oligonucleotidico
  source 1..28: mol_type = other DNA
aaaaagcaggctatggggaggcctcctt

SEQ ID NO: 62 - DNA, 31 bp, synthetic construct
  misc_feature 1..31: note = primer oligonucleotidico
  source 1..31: mol_type = other DNA
agaaagctgggttgaacatcggagacaactc

SEQ ID NO: 63 - DNA, 22 bp, synthetic construct
  misc_feature 1..22: note = primer oligonucleotidico
  source 1..22: mol_type = other DNA
caccctcagcattgactgcaca

SEQ ID NO: 64 - DNA, 23 bp, synthetic construct
  misc_feature 1..23: note = primer oligonucleotidico
  source 1..23: mol_type = other DNA
cctctcaactcactaactcactc

SEQ ID NO: 65 - DNA, 23 bp, synthetic construct
  misc_feature 1..23: note = primer oligonucleotidico
  source 1..23: mol_type = other DNA
cacctatgtgactctcaagtcac

SEQ ID NO: 66 - DNA, 23 bp, synthetic construct
  misc_feature 1..23: note = primer oligonucleotidico
  source 1..23: mol_type = other DNA
tcactcactccacctttcttcct

SEQ ID NO: 67 - DNA, 28 bp, synthetic construct
  misc_feature 1..28: note = primer oligonucleotidico
  source 1..28: mol_type = other DNA
atgggcagccatagcttcctatcttcca

SEQ ID NO: 68 - DNA, 24 bp, synthetic construct
  misc_feature 1..24: note = primer oligonucleotidico
  source 1..24: mol_type = other DNA
tcggagacaactccttcatctcct

SEQ ID NO: 69 - DNA, 29 bp, synthetic construct
  misc_feature 1..29: note = primer oligonucleotidico
  source 1..29: mol_type = other DNA
ttgtgaagttgttgacttttggagcagat

SEQ ID NO: 70 - DNA, 26 bp, synthetic construct
  misc_feature 1..26: note = primer oligonucleotidico
  source 1..26: mol_type = other DNA
tgacaattccttgttaattagaacat

SEQ ID NO: 71 - DNA, 20 bp, synthetic construct
  misc_feature 1..20: note = primer oligonucleotidico
  source 1..20: mol_type = other DNA
agaccgtgaaggttgtggac

SEQ ID NO: 72 - DNA, 20 bp, synthetic construct
  misc_feature 1..20: note = primer oligonucleotidico
  source 1..20: mol_type = other DNA
tagtgatctgccgtgtctcg

SEQ ID NO: 73 - DNA, 23 bp, synthetic construct
  misc_feature 1..23: note = primer oligonucleotidico
  source 1..23: mol_type = other DNA
aacaagggatcaataatatccca

SEQ ID NO: 74 - DNA, 22 bp, synthetic construct
  misc_feature 1..22: note = primer oligonucleotidico
  source 1..22: mol_type = other DNA
ccatgttattatctactccacc

SEQ ID NO: 75 - DNA, 22 bp, synthetic construct
  misc_feature 1..22: note = primer oligonucleotidico
  source 1..22: mol_type = other DNA
ccatgttattatctactccacc

SEQ ID NO: 76 - DNA, 21 bp, synthetic construct
  misc_feature 1..21: note = primer oligonucleotidico
  source 1..21: mol_type = other DNA
gatgatcaacagaatactcag

SEQ ID NO: 77 - protein, 280 aa, Arabidopsis thaliana
MGRPPCCDKIGIKKGPWTPEEDIILVSYIQEHGPGNWRSVPTNTGLLRCSKSCRLRWTNYLRPGIKRGNFTPHEEGMIIHLQALLGNKWASIASYLPQRTDNDIKNYWNTHLKKKLNKSDSDERSRSENIALQTSSTRNTINHRSTYASSTENISRLLEGWMRASPKSSTSTTFLEHKMQNRTNNFIDHHSDQFPYEQLQGSWEEGHSKGINGDDDQGIKNSENNNGDDVHHEDGDHEDDDDHNATPPLTFIEKWLLEETSTTGGQMEEMSHLMELSNML

SEQ ID NO: 78 - protein, 360 aa, Glycine max
MGRPPCCDKVGIKKGPWTPEEDIILVSYIQEHGPGNWRSVPTNTGLSRCSKSCRLRWTNYLRPGIKRGNFTPHEEGMIIHLQALLGNKWAAIASYLPQRTDNDIKNYWNTHLKKKLKKFQAALDPHSASDSTASGQFLPKSFSDRRSTLDISSNNNNNHGSSIRLSHHQSQSSSTTTYASSTENISRLLEGWMRSSPKQLNKGSIISQYDEDHQLQGINDDDDNNNNNFVSTTKLVNLHHLHQHKIRNKDHEQEGGGVDNNMVSHHEEFDSILSFENQNNAAWDKSTCDSMPEKSSEASAAADHHHHHHHDIAAAHERNNVRQKSENSSGAPPLSFLEKWLLDENVGHQVEEMMELSPMF

SEQ ID NO: 79 - protein, 348 aa, Glycine max
MGRPPCCDKVGIKKGPWTPEEDIILVSYIQEHGPGNWRSVPTNTGLSRCSKSCRLRWTNYLRPGIKRGNFTPHEEGMIIHLQALLGNKWAAIASYLPQRTDNDIKNYWNTHLKKKLKKFQAALDPHSASDSTASGQFLPKSFSDRRSLDISSSNNNHGSSIRLSHQSQSSSTTYASSTENISRLLEGWMRSSPKPLKGSSSQDDEDIQLQGINDDDKNNNNFESTTKLVNLHHLQHKVRNKDEHQEGGGGGVDNMVSHEEFDSILSFENLNNAAWDKSTCDSMPEKSSEAAAEYSVDHHHDIAAHERNNIRQKSENSGAPPLSFLEKWLLDESVGHQVEEMMELSPMF

SEQ ID NO: 80 - DNA, 2139 bp, synthetic construct
  source 1..2139: mol_type = other DNA
aacgctgaaacccctctctctctctctcactcactcactcgctgactttggtttccacattgactgcataattataaaggcaggaaaagagttaggtgacttagctaggcagatgaaggatggtaaaactcattagtttccttccatcaaggtcatggagtatataaagcactcacatgcattggtactatttgcaatacaagctcatctcttgggaagaaattagtttaagccttagctagaaaaaggagagtgagttagtgagttgagaggcagaagaaaagaagaagaaaggtggagtgagtgagtgaatagaaaaaaaaaacccaccatggggaggcctccttgctgtgacaaagttggcatcaagaaaggtccatggacacctgaggaggatattatccttgtctcttacatccaagaacatggtccaggaaattggagatcagtgcctactaatactggtatacctaaattcaattgtatattagtatattcagattcttagtgacttcttcttgatctatctatctatgtacatgtatgttatatattttttggtttggaatcaattatcatgacaatttgaatcaaatctttcttgaaaagggttatcaaggtgcagcaaaagttgcaggcttagatggacaaattacctaaggccaggaatcaagagagggaacttcaccccccatgaggaagggatgataattcatttgcaagctctactgggtaacaagtaatactctcaaatatatcatactttttccgtcgattttctcaacaaaaagagaatcaatattcaataccatgacatataattaaagttcacatacttttcattgattaattttgataaaaaaaattataattttagcatatgcctatctatatgatatgcctgaatttgtgaagttgtttgaattttggagcagatgggcagccatagcttcctatcttccacaaagaacagacaatgacataaagaattattggaacacacatctaaagaagaagctgaagaaatttcaagcagctttggatcctcactcagcatcagactcaacagccagtggtcaatttctaccaaagagctttagtgacagaagaagtactttagacatcagcagcaacaacaacaataatcatggctcgtccattaggcttagtcatcatcaatctcagtcctcttccacaacaacatatgcctcaagcactgagaatatctcaagactcttggaaggttggatgagatcttccccaaagcaactcaacaagggatcaataatatcccaatatgatgaagatcatcagctccaaggcatcaatgatgatgatgataataacaacaacaactttgtaagcactactaagctagtgaaccttcatcatcttcatcagcacaaaattagaaacaaagatcatgagcaagagggtggtggagtagataataacatggtttctcatcatgaagagttcgattctattttgtcctttgagaaccaaaacaatgctgcttgggacaagtccacttgtgattccatgccagaaaagagttctgaagcttctgctgctgctgatcatcatcatcatcatcatcatgatatagctgctgctcatgagaggaacaatgtcaggcaaaaatctgagaatagtagtggtgctcctccattgtcatttcttgagaagtggctcttggatgaaaacgttggtcatcaagtggaggagatgatggagttgtctccgatgttctaaattaacatggaattgggtcaccaatttaatccctttaagtttgggatgtgacattcaaatgatcatatgatattagtcagatgaaagaagagatcaaaggaatgaaattcttttgaaatctgactagggtttaatccattacaatggtggagagattcacagaagctataagggatctctttcaacttgtaacttggaagtctatgaagaatggtttaattatttcaagtaatactatatgcttagtgcatgttgctctctttgtcttgtagaaaaattacaaattaatgttaacttcagctcaaataataatactacaataacagttatattttcaagatttgaaaaaaaaattattaatttgtttggtggtaaaatgtttgc

SEQ ID NO: 81 - DNA, 2133 bp, synthetic construct
  source 1..2133: mol_type = other DNA
ctcactctctctcgctcactcgctgactttggtttccacattgactgcataattataaaggtagcaaaagagttaggtgacttagctaggtagatgaaggatggtaaaagtcttaagtttccttccatcaaagtcatggagtatataaagcactcgcatgcattggtactatttgcaagctctcttgggaagaaagtttaagccttagctagaaaaaggagagtgagtagtgagttttttgagaagcagaagaaaggaagaaaggtggagtgagtgaattcaaaaagatcacccaccatggggaggcctccttgctgtgacaaagttggcatcaagaaaggtccatggacacctgaggaggatatcatccttgtctcttacatccaagaacatggtccaggaaattggagatcagtgcctactaatactggtataacacctacttcaattatatatatcaaatccttttcttgttttgtttcttaattaactaagctgaggtgtgtgtatatatatatatatatatatatatatatatatatatatatatatatatatataatatatatatatatattatgaaagtgtcttcttcttgatctatctatgtgtatgtatattgtatacattttgctttggaatcaatcataacaatttgaatcaaatctttcttgaaaagggttatcaaggtgcagcaaaagttgcaggcttagatggacaaattacctcaggccaggaatcaagagagggaacttcactccccatgaggaaggaatgataattcacttgcaagctctactgggtaacaagtaattctcaaatatcataattttctttagttttctcaacaaagttaaagagatcaatcaataccatgataattaaagttcacatacttttcattgattaattttgacaaaaaaataactttttaatctataattttagcatatgatatgcctgaatttgtgaagttgttgacttttggagcagatgggcagccatagcttcctatcttccacaaagaacagacaatgacataaagaattattggaacacccatctaaagaagaagctgaagaaatttcaagcagccttggatccacactcagcatcagactcaacagcaagtggtcagtttctaccaaagagctttagtgacagaagaagcttagacatcagcagcagcaacaataatcatggctcatccattaggcttagtcatcagtctcagtcctcttccacaacatatgcctcaagcactgagaacatctcaagactcttggaaggttggatgagatcttccccaaagccactcaagggatcatcatcccaagatgatgaagatattcagctccaaggcatcaatgatgatgataaaaataacaacaactttgaaagcactactaagctagtgaaccttcatcatcttcagcacaaagtaagaaacaaagatgagcatcaagagggtggtggtggtggcgtagataacatggtttctcatgaagagtttgattctattttgtcttttgagaaccttaacaatgctgcttgggacaagtccacttgtgattccatgccagaaaagagttctgaagctgctgctgagtattctgttgatcatcatcatgatatagctgctcatgagaggaacaacattaggcaaaaatctgagaacagtggtgctcctccattgtcatttcttgagaagtggctcttggatgaaagtgttggtcaccaagtggaggagatgatggagttgtctcctatgttctaattaacatggaattgtcaccaattaatcccttcaagtttgggaagtgacattcaaatcttatgatattagtcagatgaaagaagagatcaaaggaatgaaactctttgagatctgactagggtttaatccatttacaatgggttgagattctcagaagctatgggatctctctttcaacttgtaacttggaagtttatgatgaagggtttaattatttcaagtaatactatatgcttagtgcatcttgctctctttgccttgtagaaaactacaaattaatgttaacttcatcagctcaaataattatacaattaataacagttatattttcaagatttgacaaaattttattaatt

SEQ ID NO: 82 - DNA, 1851 bp, Glycine max
  source 1..1851: mol_type = genomic DNA
ttaaaatagttttttttcctatcacatcattgatcttatcttatgttttttcttttctttttctactttcaatctctcccttttgttgcacaaacacaattttctctttgcaaagtgttgttttagaagcatgttcacgaaaccttcgagtagttgcatataggagatcgttatcctttcgcagaagagggagtaattaatataaatggaaagtgacagaacctaagtactaacacactatttcagtacatagcacagcttggacacatggttaagaggaactgctcgctgctcctcataaagaagcaaacactcactcactcactcactctctctctctctctctctctgcaaataatataattagatcacagcccacaaaaaaaaaatataattagaccatgaaaaagaaagttaattctcatgaaatcatgaaccagctatgtttcttcaaagaaaatcgaaccttttttcattactaataaccacttttcaagctaccctttgattattattttgcgttccattgctctaaagtagtcccatcagcccaaaaaagccgaaaagatgacacaagaggctgaaatttgacaccactcaaaagtaaaagcattgctccatatggaactatatagaacatagttacatgtcacatgactttgatataatgatatataacaatttcattttcatatgcatatataactattgattcaggagttgaattcatcatcatctttacaatctaacaagtacaaattaaatatttataatcctgcacaatataacagacaagtaaccacaaatcattattcgtttgtttcagattctctcaacattcccccaaaagccagcaacaaccttaccaaaatccctaacccctctggctatccttacagcattttctgttcctctctgtgaaaatttcatttattgttagctgtccgctaaattttggcagtaccttcatacattcaattttgcagagggtaattgaaaattaaaataaataaatcaatatctattcgttggaaaatcattaatgccataattaagtttgctttgaattttatgtttggttagtgactgacttcaattttaataaatcaaaagaatagattagaagaaaaaaatatacacttctctagaacctcaatttattagatgtcgaccaattttacatgtaagaaatgtaacatgggtgatggattagtgtgctaacgatatgaagggaaattaaagaaaagggtagcaagcaaattattaaattggtcatgatatttactataatttgattattggttaagagattaaaacaatttttttttattagaatgaacaaaattatattatttatgatttttttaatattttttatgatttttacaatcaatatttttttctttaatgttttaatcaatgtcttaaggatctgtttagtaagattcaattaaattttattaactttttggtgagagggtggcacgtgacccatcatcatagggaaaagagagagaaacgctgaaacccctctctctctctctcactcactcactcgctgactttggtttccacattgactgcataattataaaggcaggaaaagagttaggtgacttagctaggcagatgaaggatggtaaaactcattagtttccttccatcaaggtcatggagtatataaagcactcacatgcattggtactatttgcaatacaagctcatctcttgggaagaaattagtttaagccttagctagaaaaaggagagtgagttagtgagttgagaggcagaagaaaagaagaagaaaggtggagtgagtgagtgaatagaaaaaaaaaacccaccatg

SEQ ID NO: 83 - DNA, 1915 bp, Glycine max
  source 1..1915: mol_type = genomic DNA
attttcaacggaataattccctctctctctctctctttttttttagtaaaaaagtagtagaataggaagacacccaatggtaccaggcaatgctccaatgacatgctaggattgtcaatacaaatctacccatacgattattgtacacacatagatactcaactagctatagtttctaagtgttaagagacttcccaagaaaagattgttcaataagtgaaaaaagtgataagagaaattcaaacccacactaacataaattaacagtgaaaatgatcgagctatgactaaactatttgtaacatattattcatatataaatttgtttacaatactattttttttttttttaaaaaagttctatcacataatttatcttatcctacattttctttttcattttcctttcaatcttcctttgttgtgtacgtattgtacaaataaaacttctctttgcaaagcgttcttttagaagcatgttcatgaaactcaccctttgagtagttgcataggagatcgttatcctttcacagaagagggagtaattgagatcaatggaaagtgacagaacctaagtactaacacactatttcagtacatggcacagcttggacacatggttaagaggaactgctcgctgctcctcataaagaagcaaactcacgcactctctctccgtgcatataatattagaccatgaaaaagaaagtgaattctcatgaaatcatgaaccagctatttttcttcaaagaaaattgaaccttttttcattactaataaccacttttcaagctaccccttggttattttgcgttttattgctctaaagtagccccatcagcccaaaaaagccaaaaagataacacaacaggctgaaatttgacactactcgaaagtaaaagcattactccttatggaaccatatagaacaaagttccatgtcacatgaccttgatataatgatatataacaaaatcacatatgcataataatcgattcaggagttgaattcatcatctttacaagctaacaaagtacaaattaaatatttatcctgctcaatataacagacaactaaccacaaatcagtatttgtttgtttcagattctctcaacattccccaaaagccagcagcaaccttaccaaaatctccaatatccttaaggcattttctgctcctctctgtgaaaatttcatttattgttaggtgtccgctaaattttggcagtaccacacattcaattttgcagagggcaatcaaaatttaaaataaatcaaaatcgaaatcaaactattcgttggaaaatcattaatgccataattaagtttgctttgaattttatgtttggttagtgactgacttcaattttaataaatcaaaagaatagattagaagaaaaaatatacacttctccagaaccccaatttattagatgtcgaccaaatttacatgtaaaaaaatgtaacatggtgattgattagtgtgctaacgatatgaagggaaattaattcaagaaaaaggtagcaaaatattaaattttattaacttttggtgaaagggtgccacgtgacccatcaaaaaggaaaaagagagaaaaaaaaatgctggaactcctcactctctctcgctcactcgctgactttggtttccacattgactgcataattataaaggtagcaaaagagttaggtgacttagctaggtagatgaaggatggtaaaagtcttaagtttccttccatcaaagtcatggagtatataaagcactcgcatgcattggtactatttgcaagctctcttgggaagaaagtttaagccttagctagaaaaaggagagtgagtagtgagttttttgagaagcagaagaaaggaagaaaggtggagtgagtgaattcaaaaagatcacccaccatg

(19) United States
(12) Patent Application Publication
(10) Pub. No.: US 2025/0327087 A1
(43) Pub. Date: Oct. 23, 2025

(54) SOYBEAN PLANT CHARACTERIZED BY HIGH DROUGHT RESISTANCE

(71) Applicant: SIPCAM OXON S.P.A., Milano (IT)

(72) Inventors: Massimo Galbiati, LOMAGNA (LC) (IT); Piero Ciriani, Milano (IT); Chiara Tonelli, Milano (IT)

(73) Assignee: SIPCAM OXON S.P.A., Milano (IT)

(21) Appl. No.: 18/868,253

(22) PCT Filed: May 24, 2023
(86) PCT No.: PCT/EP2023/063904

§ 371 Date: Nov. 22, 2024

(30) Foreign Application Priority Data
May 25, 2022 (IT) 102022000010913

(51) Int. Cl. C12N 15/82; C12N 9/22; C12N 15/11

(52) U.S. Cl. CPC C12N 15/8273; C12N 9/226; C12N 15/111; C12N 15/8205; C12N 2310/20

(57) ABSTRACT

The invention provides a method for increasing the drought resistance of soybean plants by introducing site-specific mutations into stomatal opening genes. The invention also relates to a soybean plant, an isolated part thereof or the seeds thereof, wherein said genes have been inactivated.

The present invention relates to a method for increasing the drought resistance of soybean plants by inactivating one or both of the genes Glyma.03g006600 and Glyma.19g119300. Other aspects of the invention relate to a soybean plant, an isolated part thereof or the seeds thereof, wherein genes Glyma.03g006600 and Glyma.19g119300 have been inactivated.

### INTRODUCTION

Soybean is one of the most widespread species of agricultural interest in the world, with a cultivated area corresponding to 6% of the global agricultural area, and global annual production of over 330 million tonnes. From the economic standpoint, soy possesses strategic importance for many manufacturing industries. In the food industry, soy represents a cheap source of protein and fats, and the cheapest alternative to meat for vegetarian and vegan consumers. Soybean is also widely used in industries such as the lubricating oil, wax and paint industries.

Soybean is a very demanding crop in terms of water requirements, and its productivity is closely correlated with water availability. Even short periods of water deficiency lead to great reductions in the production of soybean grains, with losses of up to 40-50% of the produce. A low water intake adversely affects various physiological processes, including symbiotic nitrogen fixation, photosynthetic efficiency, pod setting and seed development. Plants subjected to water stress usually have a smaller number of pods per plant and seeds per pod. Moreover, the individual seeds are often small.

In recent decades there has been a worrying recurrence of drought periods, even in particularly suitable agricultural areas, with serious repercussions on soybean productivity. The ongoing climate changes will lead to intensification of such events in future, with serious effects on global soybean production. In this scenario, the selection of novel varieties characterized by a low water requirement and low production losses under water stress conditions is a necessary, urgent objective for genetic improvement of the species.

In this respect, genome editing technologies represent an innovative, strategic tool for improving soybean drought resistance, by modulating the activity of specific target genes.

### Approaches to Genetic Improvement

Various approaches have been used to improve soybean drought resistance. They include classic or assisted breeding strategies, which enable genotypes with resistance characteristics to be pre-selected. However, introgression of favorable alleles from said genotypes to elite cultivars suitable for marketing requires a great deal of time and extensive analysis of many individuals over several generations.

The development of protocols for the regeneration of multiple soybean varieties and optimization of transformation methods mediated by Agrobacterium tumefaciens has paved the way for the possibility of engineering novel soybean resistance characteristics. The majority of biotechnological applications developed to date are based on over-expression of individual genes involved in the plant's response to drought, using constitutive viral promoter CaMV35S. Said technology often gives rise to undesirable pleiotropic effects on the development and growth of the plant. For example, over-expression of MYB14 in soybeans leads to increased drought resistance, but at the same time significantly reduces the height and leaf area of the plant.

The recent design and dissemination of methods for editing the soybean genome provides an alternative method for selecting resilient novel varieties, able to maintain high production standards even under stress conditions.

### Editing Technologies

Editing technology enables the soybean genome to be modified in a precise, specifically-targeted way. The sequence of a specific target gene, and therefore its activity, can be modified by three main editing techniques: (i) Zinc-finger nuclease (ZFN), (ii) transcription activator-like effector nuclease (TALEN), and (iii) Clustered Regulatory Interspaced Short Palindromic Repeats (CRISPR)-associated Cas9. The CRISPR/Cas9 technology is more efficient and cheaper than ZFN and TALEN, and has become the preferential methodology for editing plant genomes.

The CRISPR/Cas9 system consists of three main elements: (i) the CRISPR sequence, characterized by short repeated DNA sequences alternating with spacer sequences, (ii) the Cas9 protein, containing two domains with nuclease activity (RuvC-like and HNH), and (iii) an RNA guide sequence (sgRNA) complementary to a region of the target gene, able to guide the Cas9 protein onto the target. The sgRNA sequence is therefore the element that determines the specificity of CRISPR/Cas9 for a given gene. When specific sgRNAs are used, the Cas9 protein can be directed toward a given target gene. Binding of complex sgRNA-CRISPR/Cas9 to the target gene leads to cleavage of the DNA sequence by Cas9, giving rise to the formation of a “double strand break” (DSB). Repair of the double-strand break usually involves introducing errors compared with the original gene sequence, such as insertions, deletions or base substitutions. Said new mutations induced by CRISPR/Cas9 can alter the coding sequence of the gene and prejudice its normal functions.

### Target Genes

In order for CRISPR/Cas9 technology to be applied to adapt soya to water stress, it is essential to identify the target genes involved in regulating the plant's response to water deficiency. Completion of sequencing of the entire soybean genome has allowed the identification of various genes that regulate adaptation of the plant to water stress conditions. Among said genes, transcription factors belonging to the gene families NAC, MYB, MYC, WRKY, AREB and DREB have been identified as fundamental elements for regulating the cellular, metabolic and developmental mechanisms that give rise to the soybean's stress response.

The majority of said genes improve the plant's ability to tolerate periods of drought when over-expressed in transgenic lines, using the promoter CaMV35S or other constitutive promoters. Often, however, their inactivation by CRIPR/Cas9 makes the plant more sensitive to water stress. For example, soybean plants that over-express gene Glyma.NAC8 exhibit greater drought resistance and better recovery from stress. Conversely, plants wherein gene Glyma.NAC8 has been inactivated by genome editing exhibit a lower response to water deficiency, and a high mortality rate at the end of the stress.

### Gene MYB60

AtMYB60 is a gene encoding an R2R3MYB transcription factor, specifically expressed in the stomata, wherein it governs opening of the stomatal pore in response to light and to water deficiency. The stomata are small openings on the surface of the aerial parts of terrestrial plants, surrounded by two highly specialized cells called guard cells. Opening and closing of the stomatal pore enable the plant to optimize the ratio between the CO₂, intake necessary for photosynthesis, and loss of water by transpiration. Closing of the stomata represents a first essential adaptive response by the plant to water stress conditions, enabling it to limit tissue dehydration.

Loss of the AtMYB60 function in the mutant allele atmyb60-1 leads to constitutive reduction of the stomatal opening. Even if the mutant plants are kept in the ideal growth conditions (high water availability, exposure to light), their stomata remain partly closed. Under water deficit conditions, reduced opening of the stomatal pore leads to a significant increase in the drought resistance of atmyb60-1 compared with wild-type plants.

It should be emphasized that the favorable effects of AtMYB60 inactivation on the plant's water balance are not associated with adverse effects on the plant's growth and productivity. In fact, under optimum growth conditions, the mutant atmyb60-1 does not exhibit any growth or development abnormalities, or reductions in photosynthetic efficiency, compared with wild-type plants.

### STATE OF THE ART

WO2005/085449 discloses gene constructs for selective expression of nucleic acid sequences in stomatal guard cells, in particular sequences involved in the intracellular signaling pathway modulated by abscisic acid, and in regulation of stomatal opening.

Cominelli E. et al., Current Biology vol. 15, 1196-1200 (2005), describe the characterization of Arabidopsis gene AtMYB60, as transcription factor involved in regulation of stomatal movements.

Cominelli E. et al., BMC Plant Biology 2011 (11:162), describe analysis of mutagenesis and deletion of the AtMYB60 promoter using GUS reporter-promoter systems.

The article Galbiati M. et al., BMC Plant Biology 2011, 11:142, reports the identification of gene VvMYB60 as functional orthologue of Arabidopsis gene AtMYB60, and its regulation in response to abscisic acid and to water stress conditions.

Rusconi F. et al., Journal of Experimental Botany Advance (2013), report the activity of AtMYB60 promoters in rice, tobacco and tomato, taking a reporter gene approach.

Simeoni F. et al., Scientific Report (12:533) 2022, demonstrate that AtMYB60 modulates stomatal opening by regulating oxylipin biosynthesis in the guard cells.

Simeoni F. et al., Agronomy (12:694)) 2022, report expression of gene VvMYB60 in the stomatal guard cells of grapevines, and the correlation between the levels of its expression and stomatal conductance in various grapevine genotypes.

### DESCRIPTION OF THE INVENTION

It has now been discovered that inactivation of one or both of soybean genes Glyma.19g119300 and Glyma.03g006600, by introducing site-specific mutations therein, constitutively reduces opening of the stomatal pore, enabling the plant to limit tissue dehydration and adapt to water stress conditions.

A first aspect of the invention therefore relates to a method for increasing the drought resistance of a soybean plant, which comprises inactivation of at least one target gene selected from Glyma.19g119300 and Glyma.03g006600.

Although both genes combine to control stomatal opening, gene Glyma.19g119300, whose expression is mainly localized to the stomata, is preferably inactivated. In any event, the inhibiting effect on stomatal opening of introducing mutations into one or both target genes can be modulated.

In accordance with the invention, the inactivation can relate to one or both the alleles of each gene, and can involve total or partial loss of the functionality of the corresponding encoded protein (transcription factor R2R3MYB).

In a preferred embodiment, genes Glyma.19g119300 and Glyma.03g006600 are inactivated by site-specific mutation with the CRISPR/Cas9 system.

The site-specific mutation is made possible by the action of endonuclease enzyme Cas9, combined with sgRNAs specific for the target gene. The sgRNA sequences specific for genes Glyma.03g006600 and Glyma.19g119300 are preferably selected from the group consisting of SEQ ID NO:1 to SEQ ID NO:30 and SEQ ID NO:31 to SEQ ID NO:60 respectively.

In one embodiment, the method according to the invention comprises the following steps:
(i) construction of a vector for expression of Cas9 and sgRNA in the soybean plant cell;
(ii) introduction of the expression vector into the cell of the soybean plant or a part or isolated tissue thereof, in particular a cotyledon explant, by placing said cell in contact with a culture of Agrobacterium bacteria containing the expression vector.

The method according to the invention preferably comprises the following further steps:
(iii) growing the plant containing the Cas9/sgRNA vector, and obtaining seeds;
(iv) reproduction and subsequent selection of plants with a low level of stomatal opening;
(v) further selection of plants with greater drought resistance measured under water stress conditions.

In a preferred embodiment, the vector comprises an sgRNA encoding sequence functionally bound to soya promoter U6, an expression cassette for gene Cas9, comprising a sequence encoding enzyme Cas9, preferably SpCas9, functionally bound to promoter CaMV35S, a sequence for nuclear localization of protein Cas9, and a DNA-Transfer (T-DNA) region. The expression vector can be inserted in Agrobacterium bacteria, preferably Agrobacterium tumefaciens, using techniques known to the skilled person, for example by electroporation.

In step (iv), the level of opening of the stomata can be measured by stomatal conductance analysis (gₛ) or optical microscope analysis of the dimensions of the stomatal pore.

In step (v), the plant's response to water stress conditions can be evaluated by measuring its biometric, physiological and production parameters at the various stages of the biological cycle, in plants subjected to different growth conditions wherein the water content in the medium is varied.

A further aspect of the invention relates to a soybean plant, or a part or seed thereof, wherein at least one of genes Glyma.19g119300 and Glyma.03g006600 has been inactivated, preferably gene Glyma.19g119300. The genes are preferably inactivated by introducing mutations able to suppress or reduce the functionality of the encoded protein into the respective sequences. The resulting plant can be homozygous or heterozygous for a given mutation. In a preferred embodiment, the genes are inactivated by site-specific mutation with the CRISPR/Cas9 system.

### DETAILED DESCRIPTION OF THE INVENTION

### Homology Analysis

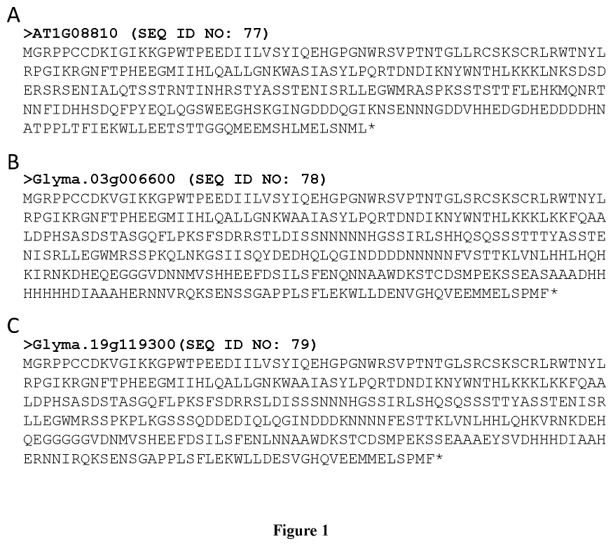

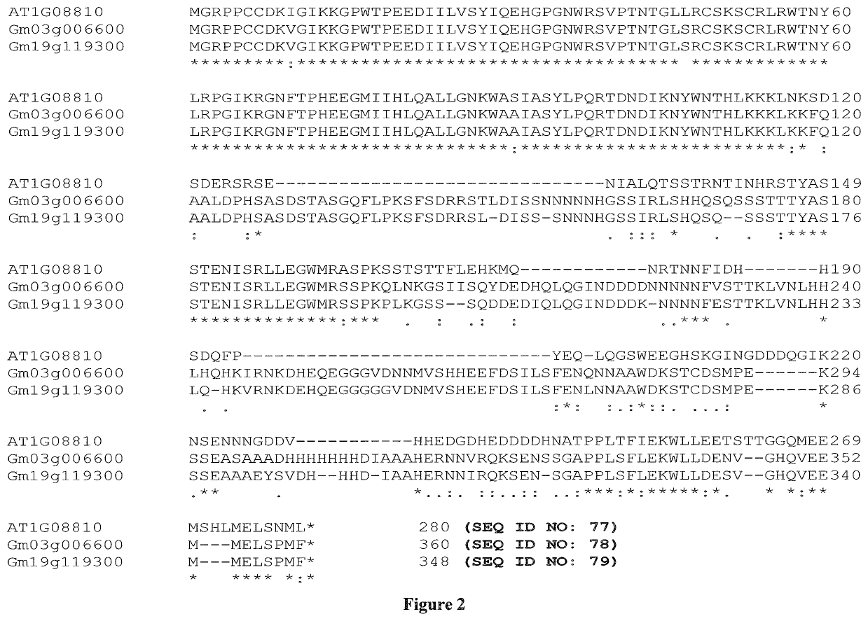

The amino-acid sequence of Arabidopsis protein AtMYB60 (FIG. 1A—SEQ ID NO: 77) was used to identify homologous proteins in the soybean genome, using BLASTp analysis (https://blast.ncbi.nlm.nih.gov). Two proteins were thus identified, named Glyma.03g006600 (FIG. 1B—SEQ ID NO:78) and Glyma.19g119300 (FIG. 1C—SEQ ID NO: 79), which are highly homologous with the Arabidopsis protein (FIG. 2).

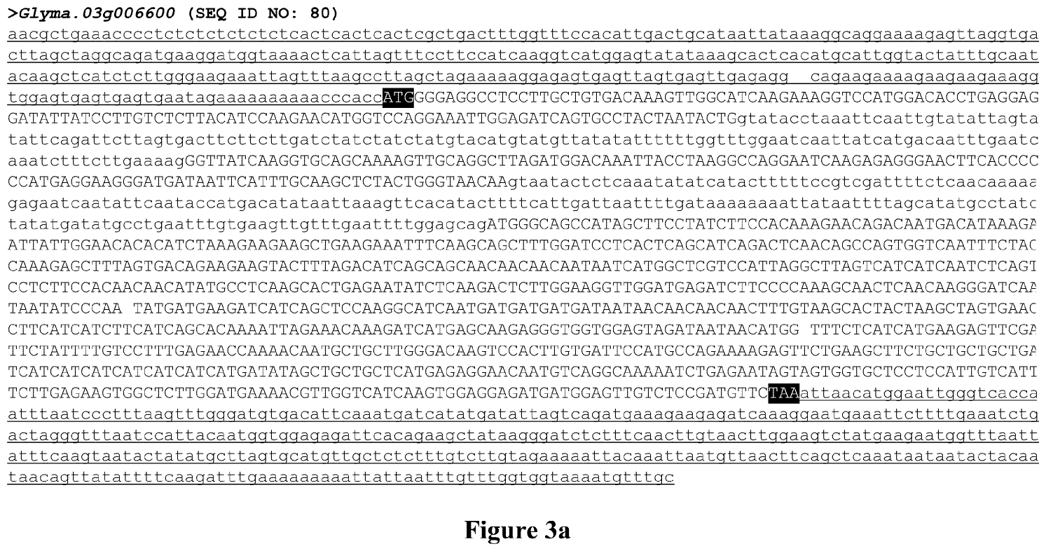

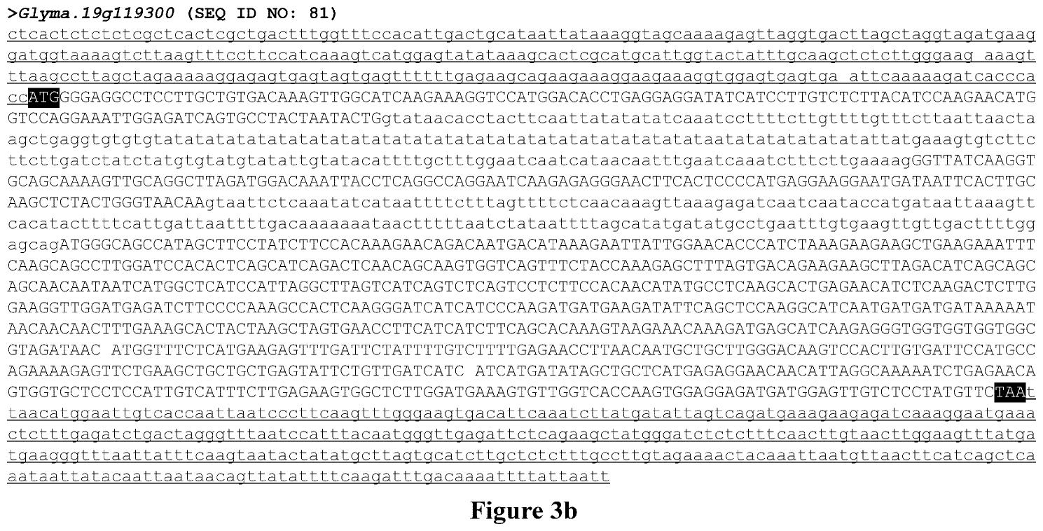

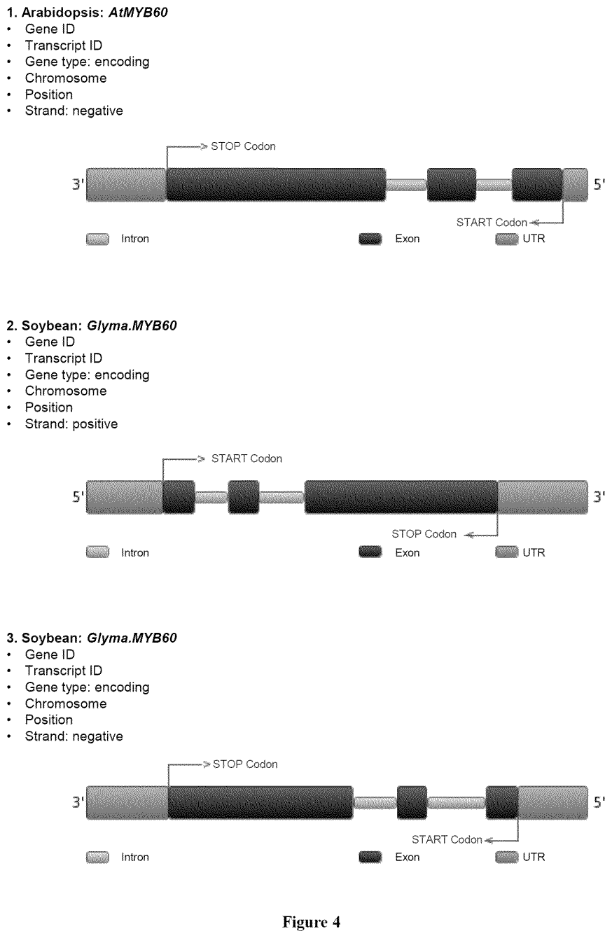

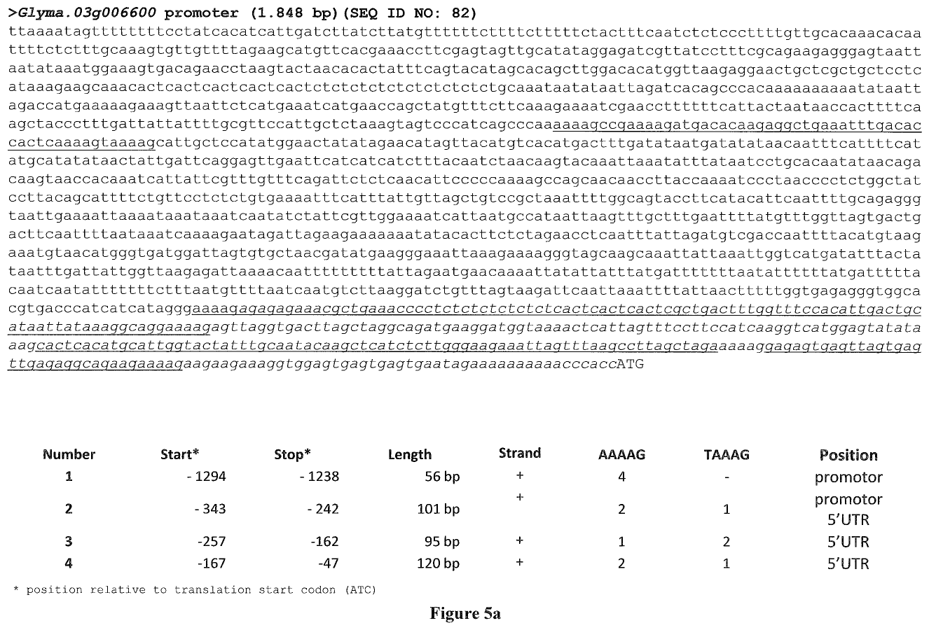

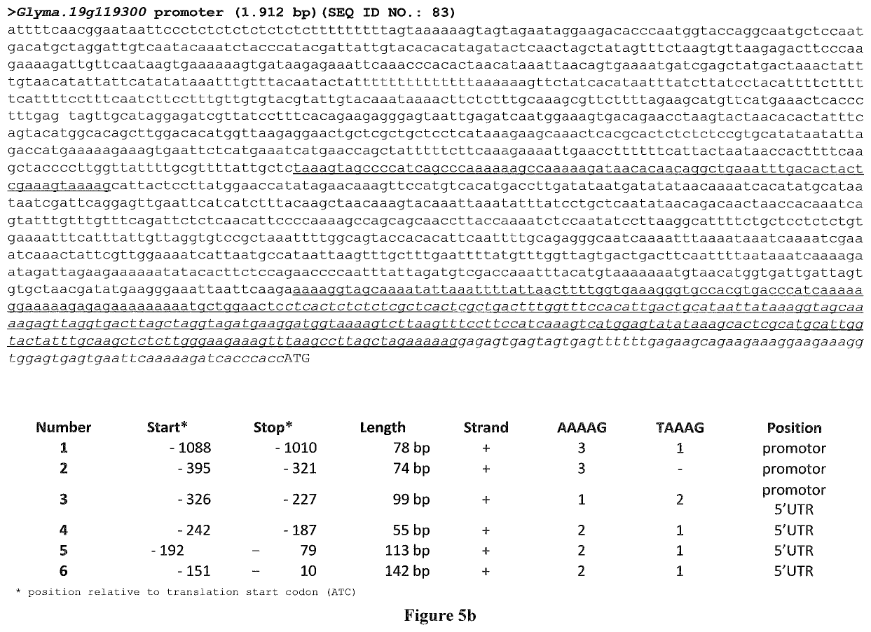

The genomic loci encoding the two proteins were mapped on soybean chromosome 3 (Glyma.03g006600, position 601548-602971, sequence deposited in GenBank Gene ID: 100802204) and soybean chromosome 19 (Glyma.19g119300, position 37492051-37493530, sequence deposited in GenBank Gene ID: 100817854) respectively. The sequence of the respective coding regions (CDS) is shown in FIG. 3. Comparative analysis of the CDS of Arabidopsis gene AtMYB60 and soybean genes Glyma.03g006600 and Glyma.19g119300 demonstrated the conservation of the gene structure in terms of introns, exons and untranslated regions (UTR) (FIG. 4).

Analysis of the genomic regions upstream of Glyma.03g006600 and Glyma.19g119300, containing the putative promoters of the two genes, demonstrated the presence of various nucleotide sequences [A/T]AAAG, corresponding to the DNA binding sites of DOF transcription factors. Said sequences, also present in the promoter of AtMYB60, represent important regulatory elements able to guide gene expression specifically in the stomata. In particular, the element in cis, which is necessary and sufficient to regulate gene expression in the stomata, consists of a cluster of at least three DOF elements distributed on the same strand in a region not exceeding 100 bp. Three DOF clusters were found in the putative promoter of Glyma.03g006600, while six clusters were mapped in the promoter of Glyma.19g119300 (FIG. 5).

### Functional Analysis

To verify the conservation of the biological function between AtMYB60 and the two soybean genes, a complementation test was performed by inserting gene Glyma.03g006600 or Glyma.19g119300 into the mutant of Arabidopsis atmyb60-1. The coding region of the two genes was amplified from cDNA obtained from soybean leaves (cv Williams 82) using the following primers:

Glyma.F3
SEQ ID NO: 61
5′-AAAAAGCAGGCTATGGGGAGGCCTCCTT-3′
Glyma.R3
SEQ ID NO: 62
5′-AGAAAGCTGGGTTGAACATCGGAGACAACTC-3′

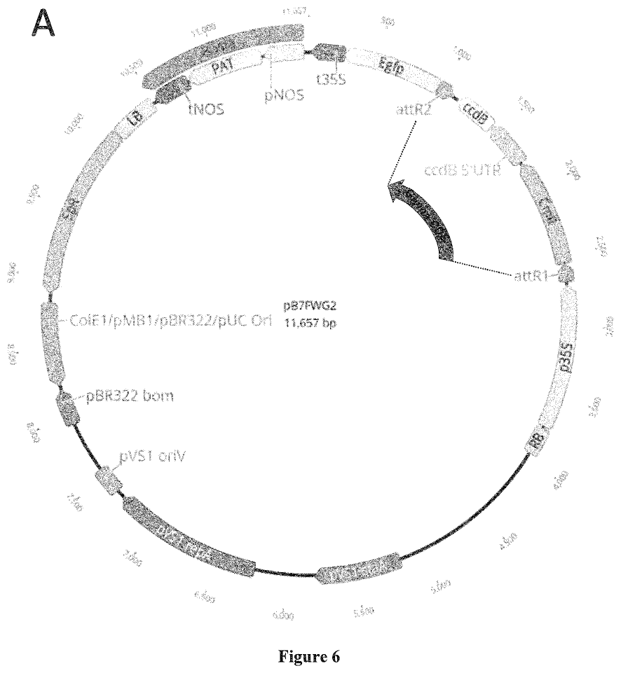

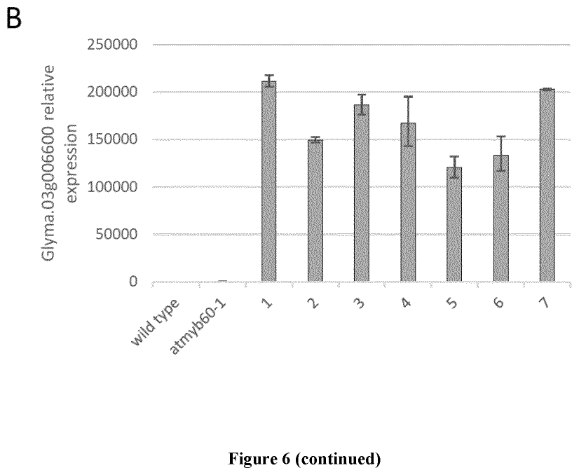

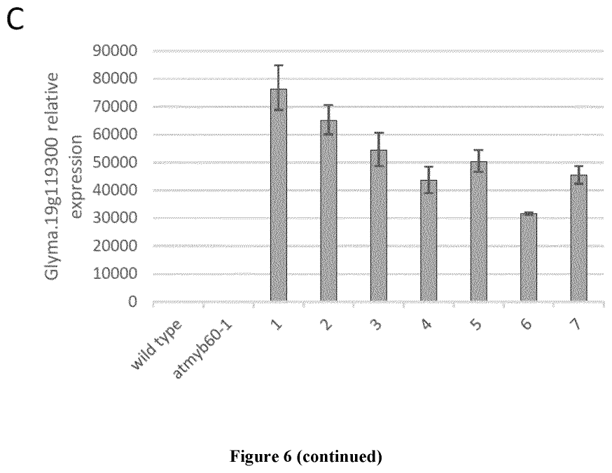

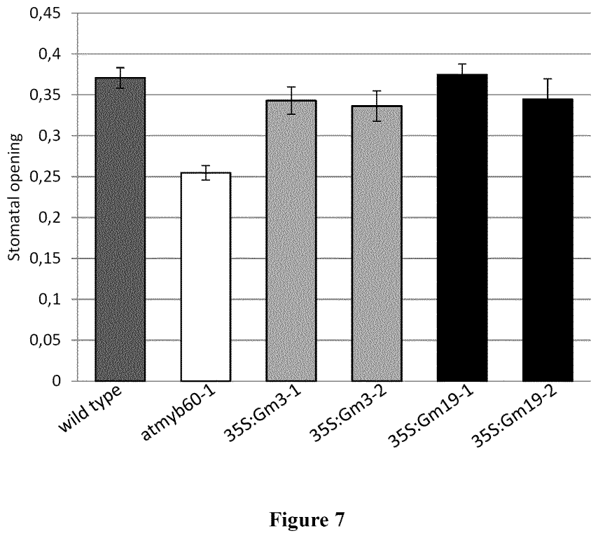

The fragments obtained were sequenced to confirm the identity of the two genes and subsequently cloned, using Gateway technology, in vector pB7FWG2, downstream of constitutive promoter CaMV35S (FIG. 6A). The two vectors containing the CDS of Glyma.03g006600 or Glyma.19g119300 were introduced into the genome of mutant atmyb60-1 by the floral dip method, using Agrobacterium tumefaciens GV3101. The transformed lines were selected with the herbicide phosphinothricin (PPT), and the resistant individuals were used for further analyses. As expected, independent lines transformed with gene Glyma.03g006600 or Glyma.19g119300 exhibited much higher expression levels of the two soybean genes than the control plants (FIGS. 6B and C). Stomatal opening was then analyzed in two independent lines per gene. Ectopic expression of both soybean genes in the Arabidopsis mutant complemented the stomatal opening defect exhibited by atmyb60-1 (FIG. 7). This demonstrates that both soybean genes are active in the stomata, wherein they act as positive regulators of stomatal pore opening, like AtMYB60. On the whole, said results confirm that Glyma.03g006600 and Glyma.19g119300 represent the functional orthologues of AtMYB60 in soybean, and support their role as targets for editing approaches designed to reduce stomatal opening in said species.

### Promoter Activity in Heterologous Systems

A particular characteristic of AtMYB60 is its expression specifically localized to the stomatal guard cells of Arabidopsis. To evaluate the cell specificity of Glyma.03g006600 and Glyma.19g119300 expression, the respective promoters were cloned from the soybean genome (cv Williams 82), fused to reporter genes GUS and GFP, and the resulting constructs were used for transient expression experiments in tobacco and for the constitution of stable transgenic lines in Arabidopsis.

The putative promoter of gene Glyma.03g006600, corresponding to the genome sequence of 1848 bp upstream of the translation start codon (FIG. 5A), was amplified with the primers:

Glyma.3F2
SEQ ID NO: 63
5′-CACCCTCAGCATTGACTGCACA-3′
Glyma.3R2
SEQ ID NO: 64
5′-CCTCTCAACTCACTAACTCACTC-3′

The putative promoter of gene Glyma.19g119300, corresponding to the genome sequence of 1912 bp upstream of the translation start codon (FIG. 5A), was amplified with the primers:

Glyma.19F2
SEQ ID NO: 65
5′-CACCTATGTGACTCTCAAGTCAC-3′
Glyma.19R2
SEQ ID NO: 66
5′-TCACTCACTCCACCTTTCTTCCT-3′

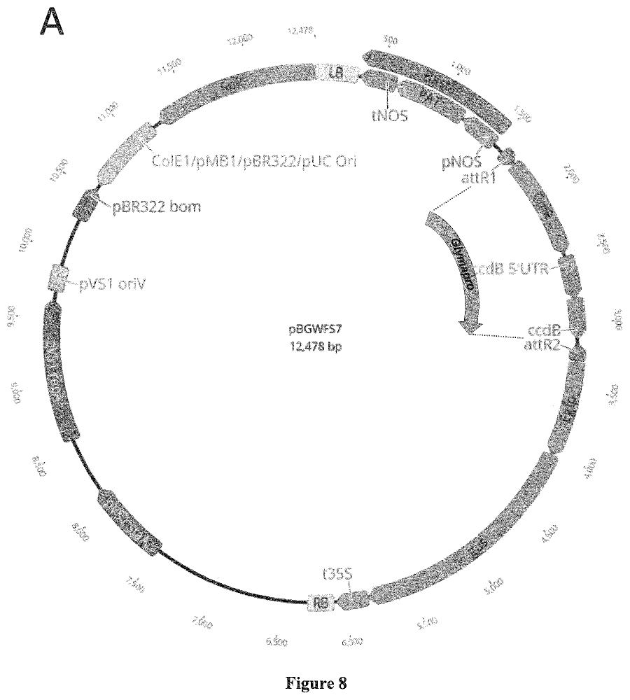

The products of amplification were cloned in vector pBGWFS7, using Gateway technology, downstream of the two reporter genes GUS and GFP (FIG. 8). The vectors thus obtained were used for a transient expression assay in tobacco leaves (Nicotiana benthamiana) infiltrated with Agrobacterium tumefaciens and subjected to GUS histochemical staining 48 hours after the agro-infiltration. N. benthamiana leaves infiltrated with the construct carrying the Glyma.03g006600 promoter mainly exhibited GUS activity in the trichomes. Conversely, leaves infiltrated with the vector containing the Glyma.19g119300 promoter mainly exhibited GUS activity in the stomatal guard cells.

The same vectors were used to produce stable lines of Arabidopsis, transformed by floral dip. The lines obtained were selected with the herbicide PPT, and their progeny analyzed by GUS histochemical assay. A total of 22 independent lines per construct were analyzed. All the lines containing the Glyma.03g006600 promoter exhibited GUS activity in the trichomes (100%). 14 (63.6%) of them also exhibited activity in the vascular tissue, while only two (9.1%) exhibited stomatal staining. 21 (95.5%) of the lines containing the Glyma.19g119300 promoter exhibited stomatal staining. 18 (81.1%) of them also exhibited GUS activity in the trichomes, whereas none exhibited staining in the vascular tissue. Interestingly, the activity of the Glyma.19g119300 promoter in Arabidopsis trichomes is very high in young leaves, and tends to decline gradually during leaf development, later localizing exclusively to the stomata of the mature leaf.

On the whole, the expression results in heterologous systems (tobacco and Arabidopsis) indicate that the promoter of gene Glyma.03g006600 is mainly active in the trichomes, whereas the activity of the promoter of Glyma.19g119300 is preferably exhibited in the stomata. This finding suggests that although both genes are able to complement the loss of function of AtMYB60 in the stomata, Glyma.19g119300 may play a more prevalent role in regulating stomatal activity than Glyma.03g006600.

### Expression in Soybean

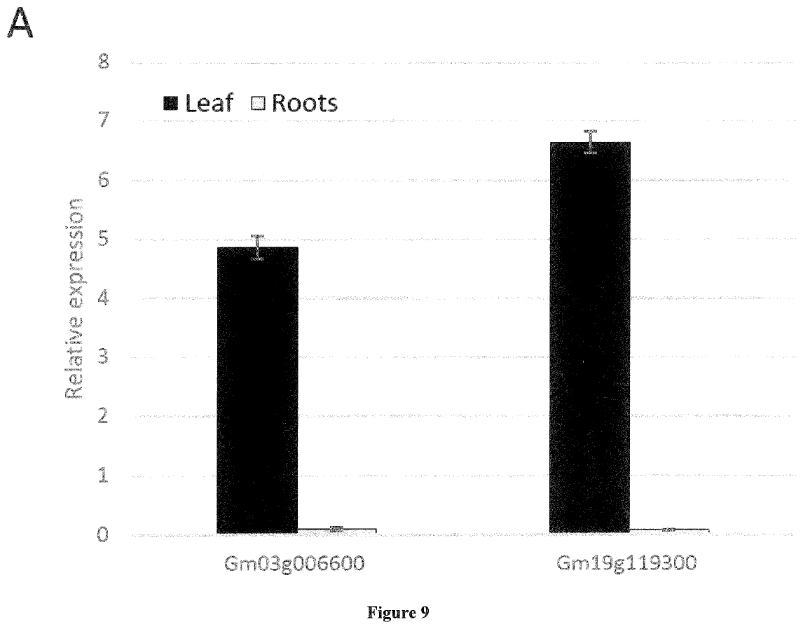

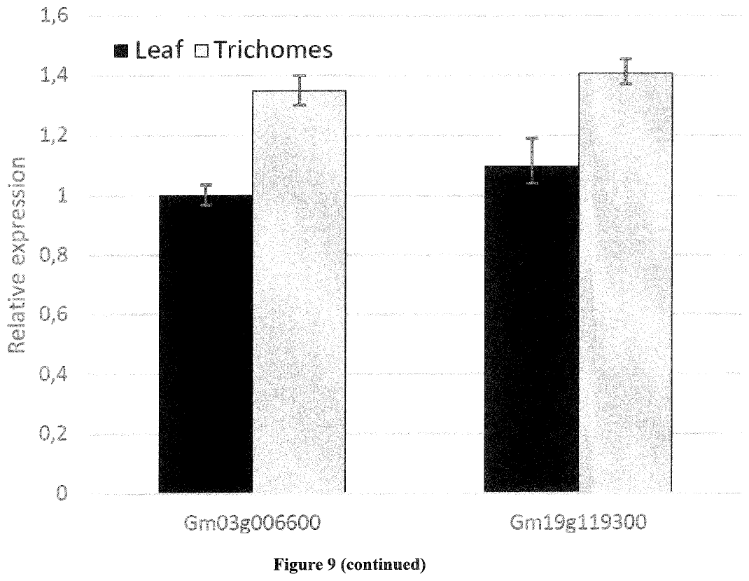

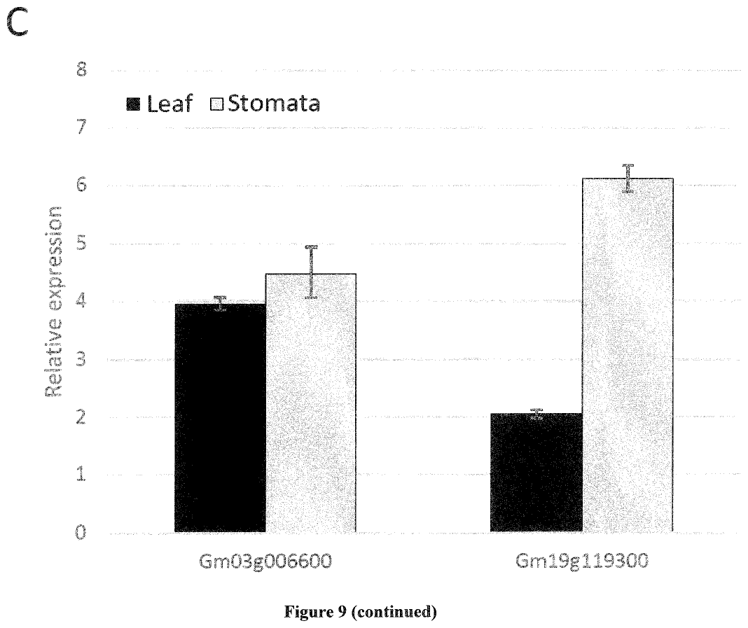

An analysis of the expression profiles of Glyma.19g119300 and Glyma.03g006600 in soybean organs and tissues was then conducted using qPCR. The analysis demonstrated that both genes are expressed in the leaves, but not the roots (FIG. 9A). Exclusive expression in the green tissues of the plant is consistent with the observations made for AtMYB60 in Arabidopsis. Expression of the two genes in whole leaves and trichomes dissected from the leaf epidermis was then compared. The comparison demonstrated that both genes are expressed, at comparable levels, in soybean trichomes (FIG. 9B). Finally, expression of Glyma.03g006600 and Glyma.19g119300 in leaves and in stomata purified from soybean leaves by successive mechanical disruption and filtration cycles was analyzed according to the methodology commonly called ice-blending. Glyma.03g006600 exhibited comparable expression levels between the whole leaf and isolated stomata, whereas Glyma.19g119300 exhibited considerably higher expression in the purified stomata than the whole leaf (FIG. 9C).

On the whole, analysis of endogenous gene expression in soybean tissues demonstrates that both are expressed in trichomes, and that Glyma.19g119300 is preferentially expressed in the stomata, consistently with the expression data obtained in heterologous systems.

### Editing of Soybean Genes

The results obtained from analysis of genes Glyma.03g006600 and Glyma.19g119300 in heterologous systems and in the soybean plant indicate a high degree of homology with Arabidopsis gene AtMYB60, in terms of sequence (nucleotides and amino acids), biological function and expression profile. In particular, Glyma.19g119300, like AtMYB60, exhibited preferential expression in the stomata.

To reduce stomatal opening and increase drought resistance in the soybean plant, a method of inactivating genes Glyma.03g006600 and Glyma.19g119300 was therefore devised. Said method is based on the CRISPR/Cas9 editing system using sgRNAs specific for genes Glyma.03g006600 and Glyma.19g119300, and involves transformation of soybean by Agrobacterium tumefaciens by integrating into the genome a T-DNA containing: (i) the bar selection marker, which confers resistance on herbicide PPT, (ii) sgRNAs specific for Glyma.03g006600 and Glyma.19g119300, and (iii) the Cas9 gene.

From the operational standpoint, the method consists of the following steps: (i) selection and synthesis of sgRNAs, (ii) constitution of pCRISPR35SCas9_BAR_sgRNA vectors, (iii) transformation of the vectors in the soybean genome by Agrobacterium tumefaciens, (iv) selection of TO edited lines, and (v) production and analysis of T1 lines.

Selection of sgRNAs

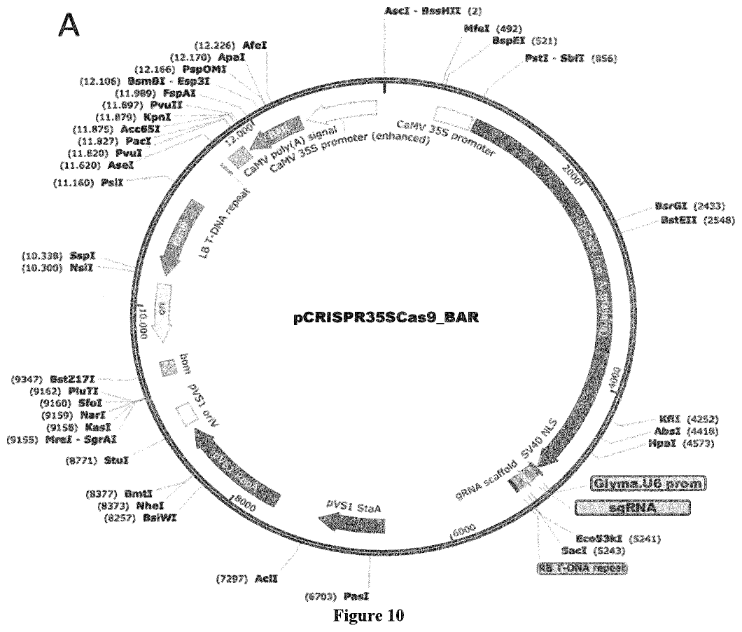

The guide sequences for CRISPR/Cas9-mediated editing were selected with the CRISPOR analysis program (http://crispor.tefor.net/). The complete CDS of Glyma.03g006600 and Glyma.19g119300 was used as target to identify the sgRNAs. The trinucleotide NGG, normally used for applications involving the CRISPR/Cas9 system of Streptococcus pyogenes, was used as Protospacer Adjacent Motif (PAM). The length of the target sequences and the corresponding sgRNAs was set at 20 base pairs (bp). The CRISPR/Cas9 vector selected for the editing experiments (pCRISPR35SCas9_BAR, FIG. 10) uses the U6 soybean promoter for sgRNA expression. Although said promoter prefers sgRNAs beginning with the G nucleotide, no filter was used for selection of the first nucleotide in the guide sequences. The initial nucleotide G is inserted in the guide sequence subsequently, when the latter is synthesized for cloning in the sgRNA-CRISPR-Cas9 vector. Of the possible sgRNAs identified by the software, 30 guides were selected for each gene, on the basis of three main criteria: (i) GC content, (ii) specificity, (iii) efficiency, and (iv) induction of out-of-frame mutations. As regards GC content, only guides with a content of not less than 20% and not more than 80% were selected, to guarantee greater cleavage efficiency. The specificity of each guide for its target was evaluated by the CFD Specificity Score method, and guides with a coefficient of specificity greater than 70 were selected. The degree of efficiency of the guides in determining cleavage of the target DNA was estimated by the method developed by Doench et al., optimized for guides expressed by U6 promoters. Guides with a degree of efficiency greater than 50 were selected. As the aim of the editing strategy was inactivation of genes Glyma.03g006600 and Glyma.19g119300, guides with an out-of-frame score greater than 50, i.e. characterized by a high probability of inducing inactivating mutations in the two targets, were selected.

A list of the guides selected for the editing experiment, the corresponding DNA sequences and the relevant information will be found in Tables 1 and 2.

TABLE 1
sgRNA guides selected for gene Glyma.03g006600.

| | | | Specificity | Efficiency | Out-of- | | SEQ |
|---|---|---|---|---|---|---|---|
| guide | guide | | cfd Spec | Doench- | Frame- | | ID |
| # | ID | Sequence | Score | Score | Score | Position | NO: |
| 1 | 132forw | AGATCAGTGCCTACTAATACTGG | 94 | 53 | 57 | HEXON 1 | 1 |
| 2 | 121rev | TTAGGTATACCAGTATTAGTAGG | 90 | 51 | 50 | HEXON 1 | 2 |
| 3 | 826rev | TTGATGATGACTAAGCCTAATGG | 91 | 50 | 71 | HEXON 3 | 3 |
| 4 | 366forw | AGGGAACTTCACCCCCCATGAGG | 88 | 67 | 73 | HEXON 2 | 4 |
| 5 | 370forw | AACTTCACCCCCCATGAGGAAGG | 87 | 53 | 80 | HEXON 2 | 5 |
| 6 | 1293forw | ATATAGCTGCTGCTCATGAGAGG | 89 | 62 | 63 | HEXON 3 | 6 |
| 7 | 739rev | CTTTGGTAGAAATTGACCACTGG | 89 | 60 | 76 | HEXON 3 | 7 |
| 8 | 862rev | GCTTGAGGCATATGTTGTTGTGG | 86 | 61 | 71 | HEXON 3 | 8 |
| 9 | 1305forw | CTCATGAGAGGAACAATGTCAGG | 89 | 47 | 56 | HEXON 3 | 9 |
| 10 | 55rev | ACAAGGATAATATCCTCCTCAGG | 88 | 53 | 82 | HEXON 1 | 10 |
| 11 | 371forw | ACTTCACCCCCCATGAGGAAGGG | 90 | 60 | 79 | HEXON 2 | 11 |
| 12 | 59forw | GAAAGGTCCATGGACACCTGAGG | 86 | 66 | 79 | HEXON 1 | 12 |
| 13 | 102forw | TACATCCAAGAACATGGTCCAGG | 87 | 50 | 70 | HEXON 1 | 13 |
| 14 | 400forw | ATTCATTTGCAAGCTCTACTGGG | 80 | 49 | 76 | HEXON 2 | 14 |
| 15 | 911forw | AATATCTCAAGACTCTTGGAAGG | 82 | 65 | 76 | HEXON 3 | 15 |
| 16 | 346forw | CTAAGGCCAGGAATCAAGAGAGG | 85 | 64 | 64 | HEXON 2 | 16 |
| 17 | 947forw | TCCCCAAAGCAACTCAACAAGGG | 88 | 64 | 75 | HEXON 3 | 17 |
| 18 | 1198rev | TTCTGGCATGGAATCACAAGTGG | 85 | 54 | 51 | HEXON 3 | 18 |
| 19 | 96forw | GTCTCTTACATCCAAGAACATGG | 84 | 62 | 83 | HEXON 1 | 19 |
| 20 | 357rev | TATCATCCCTTCCTCATGGGGGG | 82 | 67 | 72 | HEXON 2 | 20 |
| 21 | 743forw | GCATCAGACTCAACAGCCAGTGG | 85 | 72 | 72 | HEXON 3 | 21 |
| 22 | 915forw | TCTCAAGACTCTTGGAAGGTTGG | 81 | 48 | 75 | HEXON 3 | 22 |
| 23 | 946forw | TTCCCCAAAGCAACTCAACAAGG | 80 | 60 | 71 | HEXON 3 | 23 |
| 24 | 856rev | GGCATATGTTGTTGTGGAAGAGG | 74 | 52 | 73 | HEXON 3 | 24 |
| 25 | 989forw | GATGAAGATCATCAGCTCCAAGG | 82 | 60 | 57 | HEXON 3 | 25 |
| 26 | 62forw | AGGTCCATGGACACCTGAGGAGG | 80 | 71 | 63 | HEXON 1 | 26 |
| 27 | 613rev | TTGTGGAAGATAGGAAGCTATGG | 78 | 54 | 54 | HEXON 3 | 27 |
| 28 | 622rev | GTCTGTTCTTTGTGGAAGATAGG | 81 | 48 | 79 | HEXON 3 | 28 |
| 29 | 358rev | TTATCATCCCTTCCTCATGGGGG | 79 | 63 | 75 | HEXON 2 | 29 |
| 30 | 1188forw | AGAACCAAAACAATGCTGCTTGG | 77 | 52 | 59 | HEXON 3 | 30 |

TABLE 2
sgRNA guides selected for gene Glyma.19g119300.

| | | | Specificity | Efficiency | Out-of- | | SEQ |
|---|---|---|---|---|---|---|---|
| guide | guide | | cfd Spec | Doench- | Frame- | | ID |
| # | ID | Sequence | Score | Score | Score | Position | NO: |
| 1 | 912rev | AGACTGATGACTAAGCCTAATGG | 96 | 52 | 66 | HEXON 3 | 31 |
| 2 | 1016rev | TGGGATGATGATCCCTTGAGTGG | 96 | 59 | 63 | HEXON 3 | 32 |
| 3 | 1023forw | ATCTTCCCCAAAGCCACTCAAGG | 97 | 54 | 65 | HEXON 3 | 33 |
| 4 | 1024forw | TCTTCCCCAAAGCCACTCAAGGG | 96 | 62 | 68 | HEXON 3 | 34 |
| 5 | 1352forw | ATGATATAGCTGCTCATGAGAGG | 96 | 63 | 79 | HEXON 3 | 35 |
| 6 | 1197forw | TGGTGGTGGCGTAGATAACATGG | 95 | 54 | 56 | HEXON 3 | 36 |
| 7 | 1256forw | AGAACCTTAACAATGCTGCTTGG | 97 | 50 | 56 | HEXON 3 | 37 |
| 8 | 1446forw | TGAAAGTGTTGGTCACCAAGTGG | 95 | 73 | 80 | HEXON 3 | 38 |
| 9 | 1449forw | AAGTGTTGGTCACCAAGTGGAGG | 95 | 64 | 65 | HEXON 3 | 39 |
| 10 | 798rev | TGATGCTGAGTGTGGATCCAAGG | 95 | 53 | 55 | HEXON 3 | 40 |
| 11 | 1177forw | GATGAGCATCAAGAGGGTGGTGG | 77 | 60 | 40 | HEXON 3 | 41 |
| 12 | 1384forw | AGGCAAAAATCTGAGAACAGTGG | 93 | 72 | 60 | HEXON 3 | 42 |
| 13 | 121rev | GGTGTTATACCAGTATTAGTAGG | 93 | 53 | 40 | HEXON 1 | 43 |
| 14 | 945rev | AGTGCTTGAGGCATATGTTGTGG | 95 | 65 | 67 | HEXON 3 | 44 |
| 15 | 1435forw | TGGCTCTTGGATGAAAGTGTTGG | 93 | 70 | 57 | HEXON 3 | 45 |
| 16 | 939rev | TGAGGCATATGTTGTGGAAGAGG | 92 | 53 | 67 | HEXON 3 | 46 |
| 17 | 1174forw | AAAGATGAGCATCAAGAGGGTGG | 89 | 65 | 48 | HEXON 3 | 47 |
| 18 | 1170forw | AAACAAAGATGAGCATCAAGAGG | 84 | 64 | 55 | HEXON 3 | 48 |
| 19 | 1171forw | AACAAAGATGAGCATCAAGAGGG | 85 | 68 | 45 | HEXON 3 | 49 |
| 20 | 835forw | GCATCAGACTCAACAGCAAGTGG | 95 | 61 | 72 | HEXON 3 | 50 |
| 21 | 470forw | AGGGAACTTCACTCCCCATGAGG | 99 | 71 | 69 | HEXON 2 | 51 |
| 22 | 904forw | AGCAGCAGCAACAATAATCATGG | 88 | 53 | 60 | HEXON 3 | 52 |
| 23 | 1441rev | ACTCCATCATCTCCTCCACTTGG | 93 | 58 | 76 | HEXON 3 | 53 |
| 24 | 1180forw | GAGCATCAAGAGGGTGGTGGTGG | 77 | 51 | 39 | HEXON 3 | 54 |
| 25 | 1063forw | GATGAAGATATTCAGCTCCAAGG | 91 | 61 | 58 | HEXON 3 | 55 |
| 26 | 429rev | CTCTCTTGATTCCTGGCCTGAGG | 96 | 54 | 57 | HEXON 2 | 56 |
| 27 | 474forw | AACTTCACTCCCCATGAGGAAGG | 97 | 61 | 76 | HEXON 2 | 57 |
| 28 | 504forw | ATTCACTTGCAAGCTCTACTGGG | 97 | 53 | 79 | HEXON 2 | 58 |
| 29 | 55rev | ACAAGGATGATATCCTCCTCAGG | 95 | 57 | 82 | HEXON 1 | 59 |
| 30 | 463rev | ATTATCATTCCTTCCTCATGGGG | 95 | 58 | 76 | HEXON 2 | 60 |

Constitution of pCRISPR35SCas9_BAR_sgRNA Vectors

Vector pCRISPR35SCas9_BAR, containing a DNA-Transfer (T-DNA) region surrounded by two inverted terminal repeats, called “right border” (RB T-DNA repeat) and “left border” (LB T-DNA repeat), was constituted for editing Glyma.03g006600 and Glyma.19g119300. The following are comprised between the two borders: (i) an expression cassette for the bar gene, which confers resistance on the herbicide PPT, consisting of the bar encoding sequence, the CaMV35S promoter and the poly-A CaMV terminator signal, (ii) an expression cassette for the Cas9 gene, formed by the sequence encoding the SpCas9 gene optimized for expression in the plant, the CaMV35S promoter, the SV40 NLS sequence for nuclear localization of the Cas9 protein, and the poly-A CaMV terminator signal, and (iii) a scaffold-gRNA region for expression of the sgRNAs specific for Glyma.03g00890 or Glyma.19g29750 under the control of soybean promoter U6-10. The oligonucleotides corresponding to the sgRNAs were synthesized directly and cloned in the pCRISPR35SCas9_BAR plasmid using Gateway technology to generate the pCRISPR35SCas9_BAR_sgRNA vectors.

### Soybean Transformation

The method involves introducing pCRISPR35SCas9_BAR_sgRNA into the soybean genome by Agrobacterium-mediated transformation. The vectors were introduced into Agrobacterium tumefaciens strain EHA105 by electroporation, and the transformed bacteria were selected on solid LB medium with the addition of kanamycin. A single colony of Agrobacterium was used as inoculum. The Agrobacterium culture was maintained in YEB liquid culture medium until an optical density OD650 of 0.7 was reached. The bacteria were then centrifuged and resuspended in the liquid co-culture medium (CCM).

The mature soybean seeds were sterilized for about 16 hours with sodium hypochlorite-hydrochloric acid, and left to imbibe in water for about 24 hours. The seeds were then cut lengthways to separate the two cotyledons, and the outer coating was removed. The cotyledons were placed in contact with the Agrobacterium suspension for 30 minutes.

After being co-cultured with Agrobacterium, the cotyledons were transferred to Petri dishes containing CCM solid medium with the addition of PPT to select resistant explants, and kept for five days at 24° C. with a 16h light/8h dark photoperiod. The cotyledons were then transferred to SIM (+ PPT) solid medium for a total of four weeks, whereafter the explants were transferred to SEM (+ PPT) medium. When the resistant shoots reached a length of about three centimeters, they were transferred to RIM medium to promote rooting.

The seedlings obtained from the transformation/regeneration process (generation TO) were transferred to plant pots, grown in the greenhouse under controlled conditions, and analyzed to select the individuals with mutations in genes Glyma.03g00890 and Glyma.19g29750.

1. Agrobacterium LB culture medium

| | | |
|---|---|---|
| Bacto-triptone | 10 | g/L |
| Yeast extract | 5 | g/L |
| NaCl | 10 | g/L |
| bacto agar | 8 | g/L |
| kanamycin | 50 | microg/ml |
| pH 7.5 (NaOH) | | |

2. Agrobacterium YEB culture medium

| | | |
|---|---|---|
| Peptone | 10 | g/L |
| Yeast extract | 5 | g/L |
| NaCl | 5 | g/L |
| kanamycin | 50 | microg/ml |
| pH 7.5 (NaOH) | | |

3. liquid co-culture medium (CCM1)

| Gamborg B5 basal | 1/10 | |
|---|---|---|
| Gamborg vitamins 1000x | | |
| MES | 3.9 | g/L |
| sucrose | 30 | g/L |
| 6-BAP | 1.67 | mg/L |
| GA3 | 0.25 | mg/L |
| Acetosyringone | 40 | mg/L |
| dithiothreitol DTT | 154.2 | mg/L |
| pH 5.4 (KOH) | | |

4. solid co-culture medium (CCMs)

| Gamborg B5 basal | 1/10 | |
|---|---|---|
| Gamborg vitamins 1000x | | |
| MES | 3.9 | g/L |
| sucrose | 30 | g/L |
| agarose | 5 | g/L |
| 6-BAP | 1.67 | mg/L |
| GA3 | 0.25 | mg/L |
| Acetosyringone | 40 | mg/L |
| dithiothreitol DTT | 154.2 | mg/L |
| sodium thiosulphate | 158 | mg/L |
| L-cysteine | 400 | mg/l |
| pH 5.4 (KOH) | | |

5. liquid shoot-inducing medium (SIMI)

| Gamborg B5 basal | full strength | |
|---|---|---|
| Gamborg Vitamin mix | 1 | mg/L |
| MES | 0.58 | g/L |
| sucrose | 30 | g/L |
| 6-BAP | 1.67 | mg/L |
| cefotaxime | 250 | mg/L |
| carbenicillin | 250 | mg/L |
| pH 5.6 (KOH) | | |

6. solid shoot-inducing medium (SIMs)

| Gamborg B5 basal | full strength | |
|---|---|---|
| Gamborg Vitamin mix | 1 | mg/L |
| MES | 0.58 | g/L |
| sucrose | 30 | g/L |
| phytagel | 3.5 | g/L |
| 6-BAP | 1.67 | mg/L |
| cefotaxime | 250 | mg/L |
| carbenicillin | 250 | mg/L |
| Glufosinate ammonium (PPT) | 5 | mg/L |
| pH 5.6 (KOH) | | |

7. shoot elongation medium (SEM)

| MS basal | full strength | |
|---|---|---|
| Gamborg Vitamin mix | 1 | mg/L |
| MES | 0.58 | g/L |
| sucrose | 30 | g/L |
| phytagel | 3.5 | g/L |
| GA3 | 1 | mg/L |
| IAA | 0.1 | mg/L |
| Zeatin riboside | 1 | mg/L |
| asparagine | 50 | mg/L |
| glutamine | 100 | mg/L |
| cefotaxime | 250 | mg/l |
| carbenicillin | 250 | mg/L |
| PPT | 5 | mg/L |
| pH 5.6 (KOH) | | |

8. root-inducing medium (RIM)

| Gamborg B5 basal | full strength | |
|---|---|---|
| sucrose | 15 | g/L |
| MES | 0.59 | g/L |
| agar | 8 | g/L |
| Indole-butyric acid (IBA) | 1 | mg/L |
| pH 5.7 (KOH) | | |

### Selection of TO Edited Lines

TO plants grown in greenhouses are subjected to molecular analysis to verify the presence of mutations in the two genes Glyma.03g00890 and Glyma.19g29750, and to verify the presence in the genome of vector pCRISPR35SCas9_BAR_sgRNA. DNA samples are extracted from each plant, and the target regions, complementary to the sgRNAs used for editing, are amplified with the following specific primers:

Glyma.03g00890
Glyma.3F3:
SEQ ID NO: 67
5′-ATGGGCAGCCATAGCTTCCTATCTTCCA-3′
Glyma.3R3:
SEQ ID NO: 68
5′-TCGGAGACAACTCCTTCATCTCCT-3′
Glyma.19g29750
Glyma.19F3:
SEQ ID NO: 69
5′-TTGTGAAGTTGTTGACTTTTGGAGCAGAT-3′
Glyma.19R3:
SEQ ID NO: 70
5′-TGACAATTCCTTGTTAATTAGAACAT-3′

The PCR products obtained are then cloned by TA-cloning in a vector optimized for PCR fragment sequencing (e.g. PCR™4-MOUSE® TA vector). The sequences thus obtained are analyzed to verify the presence of mutations in the target regions of Glyma.03g00890 and Glyma.19g29750, and to verify the nature of the mutations.

The method involves selection of the TO plants carrying mutations that wholly or partly inactivate the activity of genes Glyma.03g00890 and Glyma.19g29750, such as frame-shift mutations, introduction of stop codons, amino-acid deletions and substitutions in relevant domains of the proteins encoded by Glyma.03g00890 and Glyma.19g29750. The TO lines selected are then analyzed to verify the presence in their genome of vector pCRISPR35SCas9_BAR_sgRNA using primers specific for the Cas9 gene:

Cas9F1:
SEQ ID NO: 71
5′-AGACCGTGAAGGTTGTGGAC-3′
Cas9R1:
SEQ ID NO: 72
5′-TAGTGATCTGCCGTGTCTCG-3′

### Production and Analysis of T1 Lines

The TO plants selected on the basis of presence of mutations having a high impact on the activity of genes Glyma.03g00890 and Glyma.19g29750 are reproduced by self-fertilization for production of T1 seeds.

### Molecular Analysis of T1 Lines

The molecular analyses conducted on the TO plants are repeated on the T1 individuals to verify the presence of the selected mutations and the heterozygous or homozygous state of the individual mutations, and the presence or absence of vector sgRNA-CRISPR/Cas9. In addition, the T1 individuals characterized by the presence of inactivating mutations are analyzed by qPCR to verify the transcript levels of genes Glyma.03g00890 and Glyma.19g29750 using the specific primers:

Glyma.03g00890
Glyma.3F4:
SEQ ID NO: 73
5′-AACAAGGGATCAATAATATCCCA-3′
Glyma.3R4:
SEQ ID NO: 74
5′-CCATGTTATTATCTACTCCACC-3′
Glyma.19g29750
Glyma.19F4:
SEQ ID NO: 75
5′-CCATGTTATTATCTACTCCACC-3′
Glyma.19R4:
SEQ ID NO: 76
5′GATGATCAACAGAATACTCAG-3′

The T1 plants carrying inactivating mutations (in the homozygous or heterozygous state) are self-fertilized to produce T2 seed and plants. The T1 individuals in whose genome vector sgRNA-CRISPR/Cas9 is present are crossed with untransformed soybean plants (cv Williams 82) to promote transgene segregation in progeny F2.

### Analysis of Stomatal Activity

The T1 lines selected undergo a series of physiological analyses to establish the effect of inactivation of Glyma.03g00890 and Glyma.19g29750 on stomatal activity. The analyses comprise measuring stomatal conductance (gₛ) using the SC-1 portable porometer manufactured by Decagon Device. The measurements are taken on five leaves per plant, repeating six measurements in each leaf, using the central portion of the lower and upper leaf surfaces. Untransformed soybean plants (cv Williams 82) are used as control for the measurements. The measurements are taken in the central part of the day (between approximately 11 a.m. and 1 p.m.) in plants exposed to light (light intensity 400 μM m-² s⁻¹) for at least four hours.

Measurements of stomatal opening in fragments of epidermis obtained from the leaves of edited plants and control plants are taken in parallel. The fragments are taken from leaves of plants adapted to darkness, incubated in a solution of KCl 30 mM, MES-KOH 10 mM, pH 6.5, and kept in the dark or exposed to light (400 μM m-² s⁻¹) for four hours. The stomata are then photographed under the optical microscope (40× enlargement), and the images are analyzed with ImageJ software (https://imagej. net/) to measure the width and length of the stomatal orifice. The degree of stomatal opening is calculated as the ratio between the width and length of the stomatal orifice.

Finally, the rate of water loss in cut leaves is evaluated to assess the transpiration rate of the T1 plants. Four leaves, of comparable developmental stage and size, are taken from control plants and edited plants and left to dry in a controlled environment at a temperature of 25° C. and relative humidity of 50%. The weight of each leaf is determined at regular intervals for four hours, and the water loss was expressed as a percentage of the initial fresh weight.

The analyses described, taken as a whole, allow the selection of T1 individuals characterized by a low level of opening of the stomatal pore, low stomatal conductance and low transpiration. Said individuals were further selected for the production of T2 seeds and plants, by means of self fertilization.

### Analysis of Drought Response in Selected T2 Lines

Each T2 line is analyzed again to confirm the presence of the selected mutation in progenitors TO and T1 and to confirm the absence of vector sgRNA-CRISPR/Cas9.

The T2 lines then undergo physiological analysis to evaluate the plant's response to water stress conditions. Untransformed plants and T2 lines are grown in plant pots under three different growing conditions: (i) control condition wherein the soil is maintained at a relative water content of 80% of field capacity (FC), (ii) moderate stress condition, wherein the soil is maintained at 50% FC, and (iii) high stress conditions, wherein the soil is maintained at 30% FC.

The performance of the various T2 lines (and of the untransformed control plants) under the three water conditions is determined by measuring various biometric, physiological and production parameters at the various stages of the biological cycle.

The biometric parameters considered comprise the number of internodes produced, the length of the internodes, the total height of the stem at maturity, and the average size of the leaf blade.

The physiological parameters comprise (i) measuring stomatal conductance and photosynthetic efficiency (Aₙ, mmol CO₂ m⁻² s⁻¹), determined with the LI-6400 portable system (Li-Cor Inc., Lincoln, NE, USA), and (ii) measuring the leaf water potential (Ψleaf, MPA), determined with a Scholander pressure chamber (model PMS-1000, PMS Instruments, Corvallis, OR, USA).

The production parameters comprise the flowering period, number of pods produced, number of seeds per pod, mean weight of the seeds, and total weight of the seeds produced.

The T2 lines that exhibit the best response to water deficiency, evaluated on the basis of the parameters listed above, are selected and constitute the genetic starting material for breeding programs designed to introgress mutations in genes Glyma.03g00890 and Glyma.19g29750 into elite soybean cultivars in order to develop novel varieties characterized by high drought resistance.

1. A method for increasing the drought resistance of a soybean plant, which comprises inactivating at least one target gene selected from Glyma.19g119300 and Glyma.03g006600 in said plant.

2. The method according to claim 1, wherein said target gene is Glyma.19g119300.

3. The method according to claim 1, wherein the target gene is inactivated by inserting a site-specific mutation through Cas9 enzyme nuclease activity in combination with an sgRNA specific for said target gene.

4. The method according to claim 3, wherein said sgRNA specific for the target gene Glyma.19g119300 is encoded by a nucleotide sequence selected from SEQ ID NO:31 through SEQ ID NO:60.

5. The method according to claim 3, wherein said sgRNA specific for the target gene Glyma.03g006600 is encoded by a nucleotide sequence selected from SEQ ID NO:1 through SEQ ID NO:30.

6. The method according to claim 3, which comprises the following steps:
(i) constructing an expression vector able to express a Cas9 enzyme and an sgRNA in a soybean plant cell;
(ii) introducing the expression vector into a cell from the soybean plant, or a part or isolated tissue thereof by contacting said cell with a culture of Agrobacterium bacteria containing said expression vector.

7. The method according to claim 6, wherein said expression vector comprises:
the sgRNA coding sequence functionally linked to the U6 soybean promoter;
an expression cassette for the Cas9 gene, comprising a sequence encoding the Cas9 enzyme, the CaMV35S promoter and a sequence for the nuclear localization of the Cas9 protein; and
a DNA-Transfer region.

8. The method according to claim 6 further comprising:
(iii) growing the plant containing the Cas9/sgRNA expression vector and obtaining the seeds;
(iv) growing the plants generated by the seeds and subsequently selecting the plants with a reduced stomatal aperture level;
(v) further selecting the plants with increased resistance to drought by testing the plants under water stress conditions.

9. A soybean plant, or a part or seed thereof, wherein at least one of the Glyma.19g119300 and Glyma.03g006600 genes has been inactivated.

10. The soybean plant, part or seed thereof according to claim 9, wherein said gene is Glyma.19g119300.

11. The soybean plant, part or seed thereof according to claim 9, wherein the inactivation of at least one of said Glyma.19g119300 and Glyma.03g006600 genes is obtained by means of site-specific mutation with the CRISPR/Cas9 system.

12. The soybean plant, part or seed thereof according to claim 11, which is homozygous or heterozygous for said mutation.

13. The method according to claim 6, wherein said a part or said isolated tissue of the soybean plant is a cotyledon explant.
